(19) United States
(12) Patent Application Publication
(10) Pub. No.: US 2025/0191705 A1
(43) Pub. Date: Jun. 12, 2025

(54) DIGITAL MICROSCOPY DATA VISUALIZATION SYSTEMS AND METHODS FOR USING THE SAME

(71) Applicant: IDEXX Laboratories, Inc., Westbrook, ME (US)

(72) Inventors: Jeremy Hammond, Standish, ME (US); Heidi Peta, Edmonton (CA); Laura Snyder, Asheville, NC (US); Amy Valenciano, Mound, TX (US); Jim Russell, North Yarmouth, ME (US)

(73) Assignee: IDEXX Laboratories, Inc., Westbrook, ME (US)

(21) Appl. No.: 18/968,352

(22) Filed: Dec. 4, 2024

Related U.S. Application Data
(60) Provisional application No. 63/606,820, filed on Dec. 6, 2023.

(51) Int. Cl. G16H 10/40; G06V 10/776; G06V 20/69; G16H 50/20

(52) U.S. Cl. CPC G16H 10/40; G06V 10/776; G06V 20/698; G16H 50/20

(57) ABSTRACT

A method is disclosed including receiving an image of a plurality of cells of a biological sample; identifying, by a processor executing an image recognition machine-learning logic on the image, one or more cells of the plurality of cells as comprising one or more attributes associated with a condition; extracting individual images of the one or more identified cells; determining diagnostic data comprising one or more identifiable parameters associated with the plurality of cells; determining whether the one or more identifiable parameters are associated with the first condition; and displaying the individual images and the diagnostic data.

### CROSS REFERENCE TO RELATED APPLICATION

This application claims priority to U.S. Provisional Patent Application Ser. No. 63/606,820, filed on Dec. 6, 2023, entitled “Digital Microscopy Data Visualization Systems and Methods for Using the Same”, the entire contents of which are incorporated by reference in the present disclosure.

### TECHNICAL FIELD

The present specification relates to cytology, and more particularly, to digital microscopy data visualization systems and methods for using the same.

### BACKGROUND

Microscopy may be utilized to interpret cells in biological samples. In particular, machine learning and artificial intelligence techniques may be used to automatically analyze cells and determine cell attributes that may indicate various disease states. However, multiple types of analysis may be performed on biological samples. Accordingly, a need exists for improved digital microscopy data visualization.

### SUMMARY

In one embodiment, a method includes receiving an image of a plurality of cells of a biological sample, identifying, by a processor executing an image recognition machine-learning logic on the image, one or more cells of the plurality of cells as comprising one or more attributes associated with a condition, extracting individual images of the one or more identified cells, determining diagnostic data based at least in part on the one or more attributes, the diagnostic data comprising one or more identifiable parameters, and displaying the individual images and the diagnostic data.

In another embodiment, an apparatus comprises a processor configured to receive an image of a plurality of cells of a biological sample, identify one or more cells of the plurality of cells as comprising one or more attributes associated with a condition, extract individual images of the one or more identified cells, determine diagnostic data based at least in part on the one or more attributes, the diagnostic data comprising one or more identifiable parameters, and display the individual images and the diagnostic data.

### BRIEF DESCRIPTION OF THE DRAWINGS

The embodiments set forth in the drawings are illustrative and exemplary in nature and are not intended to limit the disclosure. The following detailed description of the illustrative embodiments can be understood when read in conjunction with the following drawings, where like structure is indicated with like reference numerals and in which:

FIG. 1 schematically depicts a digital microscope, according to one or more embodiments shown and described herein;

FIG. 2 schematically depicts an electronic control unit (ECU) of the digital microscope of FIG. 1, according to one or more embodiments shown and described herein;

FIG. 3 schematically depicts memory modules of the electronic control module of FIG. 2, according to one or more embodiments shown and described herein;

FIG. 4 depicts a flowchart of an example method for operating the digital microscope of FIG. 1, according to one or more embodiments shown and described herein;

FIG. 5 illustrates an example image that may be output by the digital microscope of FIG. 1, according to one or more embodiments shown and described herein;

FIG. 6 illustrates another example image that may be output by the digital microscope of FIG. 1, according to one or more embodiments shown and described herein; and

FIG. 7 illustrates another example image that may be output by the digital microscope of FIG. 1, according to one or more embodiments shown and described herein.

### DETAILED DESCRIPTION

Microscopy has long been an important tool for clinical pathologists to interpret cells in biological samples, such as blood samples, fine needle aspirates, lymph node aspirates, body cavity fluids, urine, and the like. Microscopy traditionally involved a clinical pathologist using multiple fields of view to visually quantify different features of cells of a biological sample to classify the cells and provide guidance around disease states that are present in the biological sample.

However, machine learning and artificial intelligence techniques may be used to perform automated analysis of biological samples, as disclosed herein. In particular, a digital microscope may capture images of a blood sample. A first algorithm may utilize machine learning and artificial intelligence techniques to identify individual cells in a blood sample and determine attributes of the identified cells. The determined attributes of the identified cells may indicate a condition or disease state. While reference is made herein to analysis of blood samples, it should be understood that in other examples, the techniques disclosed herein may be used for other types of biological samples, such as fine needle aspirates, lymph node aspirates, body cavity fluids, urine, and the like.

In embodiments, a second algorithm determines diagnostic data, such as statistics associated with the cells of the blood sample. This diagnostic data may also indicate a condition or disease state. The images of the cells and the diagnostic data may then be displayed to a user (e.g., a clinical pathologist). By displaying both the images and the diagnostic data, the user may be able to quickly discern a disease state. Furthermore, by analyzing the biological sample with two different approaches, the performance of each algorithm can be confirmed by the other algorithm. Further, by presenting two different types of data to the user (images and diagnostic data), the user may ensure that the presentation of a disease state is consistent between the two algorithms, thereby providing confidence in the algorithms.

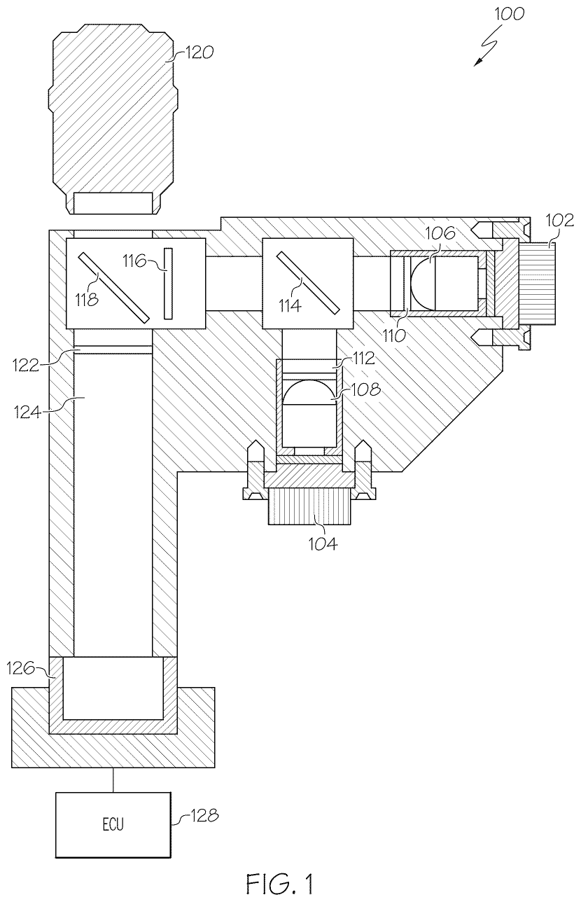

Turning now to FIG. 1, an exemplary digital microscope 100 is schematically shown. The digital microscope 100 includes a fluorescent blue light source 102, a fluorescent ultraviolet light source 104, collector lenses 106 and 108, a blue excitation filter 110, an ultraviolet excitation filter 112, an excitation dichoric 114, a field lens 116, an imaging dichoric 118, an objective lens 120, a triband filter 122, a tube lens 124, a camera 126, and an electronic control unit (ECU) 128. In some examples, the digital microscope 100 may include a brightfield source above the objective lens 120. While the digital microscope 100 includes the fluorescent blue light source 102, the fluorescent ultraviolet light source 104, the collector lenses 106 and 108, the blue excitation filter 110, the ultraviolet excitation filter 112, the excitation dichoric 114, the field lens 116, the imaging dichoric 118, the objective lens 120, the triband filter 122, the tube lens 124, and the camera 126, it should be understood that this is merely an example, and digital microscopes according to the present disclosure can have any suitable construction and configuration. For example, while the digital microscope 100 is depicted as an inverted microscope, it should be understood that the digital microscope 100 could be an upright microscope.

In operation, a blood sample is stained, provided on a carrier, and placed along the path of the fluorescent blue light source 102, the fluorescent ultraviolet light source 104, and/or the brightfield source. One of the fluorescent blue light source 102, the fluorescent ultraviolet light source 104, or the brightfield source illuminates the sample, and an image of the sample is captured by the camera 126. The image captured by the camera 126 is transmitted to the ECU 128 for automated analysis, as disclosed herein. In particular, the ECU 128 may analyze the image captured by the camera 126 using two different algorithms, and present the data in a user-friendly way, as disclosed in further detail below.

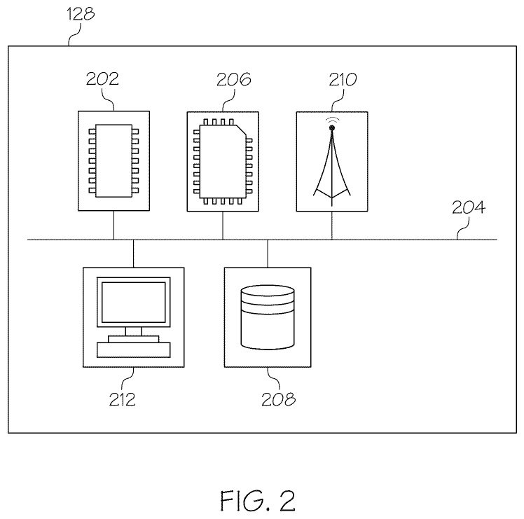

FIG. 2 schematically depicts an example configuration of the ECU 128 of FIG. 1. In the illustrated example, the ECU 128 includes one or more processors 202, a communication path 204, one or more memory modules 206, a data storage component 208, network interface hardware 210, and an output device 212, the details of which will be set forth in the following paragraphs.

Each of the one or more processors 202 may be any device capable of executing machine readable and executable instructions. Accordingly, each of the one or more processors 202 may be a controller, an integrated circuit, a microchip, a computer, or any other physical or cloud-based computing device local to or remote from the digital microscope. The algorithms, including the trained models, signal preprocessing, and noise removal methods discussed below, may be executed by the one or more processors 202. The one or more processors 202 are coupled to a communication path 204 that provides signal interconnectivity between various modules of the ECU 128. Accordingly, the communication path 204 may communicatively couple any number of processors 202 with one another, and allow the modules coupled to the communication path 204 to operate in a distributed computing environment. Specifically, each of the modules may operate as a node that may send and/or receive data. As used herein, the term “communicatively coupled” means that coupled components are capable of exchanging data signals with one another such as, for example, electrical signals via conductive medium, electromagnetic signals via air, optical signals via optical waveguides, and the like.

Accordingly, the communication path 204 may be formed from any medium that is capable of transmitting a signal such as, for example, conductive wires, conductive traces, optical waveguides, or the like. In some embodiments, the communication path 204 may facilitate the transmission of wireless signals, such as WiFi, Bluetooth®, Near Field Communication (NFC), and the like. Moreover, the communication path 204 may be formed from a combination of mediums capable of transmitting signals. In one embodiment, the communication path 204 comprises a combination of conductive traces, conductive wires, connectors, and buses that cooperate to permit the transmission of electrical data signals to components such as processors, memories, sensors, input devices, output devices, and communication devices. Additionally, it is noted that the term “signal” means a waveform (e.g., electrical, optical, magnetic, mechanical, or electromagnetic), such as DC, AC, sinusoidal-wave, triangular-wave, square-wave, vibration, and the like, capable of traveling through a medium.

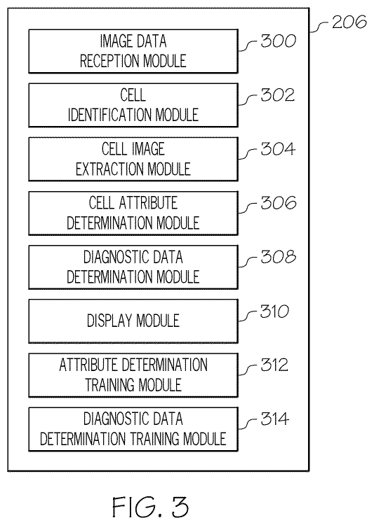

The ECU 128 includes one or more memory modules 206 coupled to the communication path 204. The one or more memory modules 206 may comprise RAM, ROM, flash memories, hard drives, or any device capable of storing machine readable and executable instructions such that the machine readable and executable instructions can be accessed by the one or more processors 202. The machine readable and executable instructions may comprise logic or algorithm(s) written in any programming language of any generation (e.g., 1GL, 2GL, 3GL, 4GL, or 5GL) such as, for example, machine language that may be directly executed by the processor, or assembly language, object-oriented programming (OOP), scripting languages, microcode, etc., that may be compiled or assembled into machine readable and executable instructions and stored on the one or more memory modules 206. Alternatively, the machine readable and executable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components. The memory modules 206 are discussed in more detail below in connection with FIG. 3.

Referring still to FIG. 2, the example ECU 128 includes the data storage component 208. The data storage component 208 may store data captured by the digital microscope 100, as disclosed in further detail below. The data storage component 208 may also store other data used by the various components of the ECU 128.

Referring still to FIG. 2, the ECU 128 comprises network interface hardware 210 for communicatively coupling the digital microscope 100 to one or more external devices. For example, data may be transferred to and processed by an external computing device. This may reduce the computational load on the ECU 128. However, in some examples, the network interface hardware 210 may be omitted.

Referring still to FIG. 2, in some embodiments, the ECU 128 comprises an output device 212. The output device 212 may include a graphical user interface (GUI), a screen, one or more devices in communication with the one or more processors 202 (such as smartphones, tables, and the like), and/or any other device or interface suitable for displaying data. In some examples, the output device 212, or another device, may be configured to as an input device to receive user input. The output device 212 may display images captured by the digital microscope 100 or diagnostic data determined by the ECU 128. While in the embodiment depicted in FIG. 2, the output device 212 is a component of the ECU 128 of the digital microscope 100 (FIG. 1), it should be understood that this is merely an example, and in some embodiments, the output device 212 is communicatively coupled to the ECU 128.

Referring now to FIG. 3, the one or more memory modules 206 include an image data reception module 300, a cell identification module 302, a cell image extraction module 304, a cell attribute determination module 306, a diagnostic data determination module 308, a display module 310, an attribute determination training module 312, and a diagnostic data determination training module 314. Each of the image data reception module 300, the cell identification module 302, the cell image extraction module 304, the cell attribute determination module 306, the diagnostic data determination module 308, the display module 310, the attribute determination training module 312, and the diagnostic data determination training module 314 may be a program module in the form of operating systems, application program modules, and other program modules. Such a program module may include, but is not limited to, routines, subroutines, programs, objects, components, data structures, and the like for performing specific tasks or executing specific data types as will be described below.

The image data reception module 300 may receive one or more images of a biological sample captured by the digital microscope 100. In particular, the image data reception module 300 may receive an image captured by the camera 126 when a biological sample is illuminated by the fluorescent blue light source 102, the fluorescent ultraviolet light source 104, and/or the brightfield light source. As such, the image data reception module 300 may receive one or more images of a plurality of cells of a biological sample. In some examples, the image data reception module 300 may receive multiple images of a biological sample captured by the camera 126 as the fluorescent blue light source 102, the fluorescent ultraviolet light source 104, and/or the brightfield light source illuminates the biological sample with different wavelengths of light. This may allow for different properties of cells to be determined, as different cell types may respond in different ways to illumination by different wavelengths of light.

Referring still to FIG. 3, the cell identification module 302 may identify one or more cells contained in the biological sample based on the one or more images received by the image data reception module 300, as disclosed herein. In particular, the cell identification module 302 may execute image recognition machine-learning logic on the received images to identify one or more cells. In embodiments, a variety of machine-learning techniques may be used to train the cell identification module 302 to define bounding boxes around individual cells in the images of biological samples received by the image data reception module 300. This may allow individual cells to be analyzed, as disclosed in further detail below. In some examples, the cell identification module 302 may identify particular images received by the image data reception module 300 having the best focus and execute the image recognition machine-learning logic on those identified images. This may improve the performance of the cell identification module 302 by ensuring that the best quality images are analyzed.

Referring still to FIG. 3, the cell image extraction module 304 may extract individual images of the one or more cells identified by the cell identification module 302. In particular, the cell image extraction module 304 may extract individual images of each cell defined by a bounding box by the cell identification module 302, as discussed above. This may allow for further analysis of the individual cell images, as disclosed in further detail below.

Referring still to FIG. 3, the cell attribute determination module 306 may determine attributes of the cells based on images of the cells extracted by the cell image extraction module 304. In particular, the cell attribute determination module 306 may classify the cells based on the images of the cells. Machine learning techniques may be used to train the cell attribute determination module 306 to classify the cells based on cell type and cell attributes. For example, kernel images may be defined as reference images for classification. An image of a cell may then be scanned to find areas where there is a strong match between the pixels in the cell image and the pixels in a reference kernel image. In particular, a machine learning model (e.g., a convolutional neural network) may be trained to classify cell images using supervised learning techniques with the reference kernel images used as ground truth data. After the machine learning model is trained, the cell attribute determination module 306 may use the trained model to classify the cell based on the reference kernel image it most closely matches. In some examples, the cell attribute determination module 306 may also determine a confidence value indicating how closely the cell image matches the reference kernel image, thereby providing a confidence level of the classification. For example, the cell attribute determination module 306 may output a confidence level of a disease state associated with a cell image.

In embodiments, the cell attribute determination module 306 may classify the cells based on cell type (e.g., red blood cells, white blood cells, platelets, epithelial cells, round cells, inflammatory cells, and the like). The cell attribute determination module 306 may also classify the cells based on attributes such as abnormalities or disease states (e.g., large cell lymphoma, left shift).

In particular, the specific classification performed by the machine learning-based for morphology analysis depends on the diagnostic objective and the types of structures being analyzed. Some examples of common classifications in medical diagnostics include:
Normal vs. abnormal cells: The model distinguishes between normal cells and cells exhibiting abnormal morphology, such as cancerous or diseased cells. This classification helps identify potential pathological conditions.
Morphologic cell assessment:
   Detection of overall presentation of cell; and
   Detection of cell objects and features, such as nuclear features, vacuoles, organisms, inclusions, and the like.
Cell type identification: The model classifies cells into different cell types based on their morphology and/or size. For example, in blood cell analysis, the model can classify red blood cells, white blood cells, and platelets.
Subcellular component identification: The model identifies and classifies specific subcellular components or organelles within cells. This can include classifying nucleus, cytoplasm, mitochondria, or other cellular structures.
Differentiation of cell stages: The model determines the stage or maturation level of cells.
Parasite detection: The model detects eggs, ova, parasites, bacteria, or the like in a sample, such as an ear swab sample or fecal sample.
Disease-specific classification: The model classifies cells based on specific diseases or conditions. For example, in histopathology, the model can classify cells based on different types of cancer.

The algorithms residing in the cell attribute determination module 306 may utilize pre-trained machine learning models to analyze the data and provide diagnostic information. Generally, these machine learning models are developed (trained and tested) at centralized locations that collect large amounts of patient data from multiple sources (including, for example, multiple point of care systems) and have extensive computing resources to perform the necessary training and testing to generate the machine learning models.

Referring still to FIG. 3, the diagnostic data determination module 308 may determine diagnostic data associated with the biological sample, as disclosed herein. The diagnostic data determined by the diagnostic data determination module 308 may be associated with multiple cells of the biological sample (e.g., statistics associated with the cells). The determined diagnostic data may comprise one or more identifiable parameters (e.g., size, circularity, contrast, uniformity, and the like) that can be mathematically derived from one or more cells in the images extracted by the cell image extraction module 304. In particular, the diagnostic data determination module 308 may determine particular diagnostic data associated with the biological sample that confirms a disease state or other attributes determined by the cell attribute determination module 306. In some examples, the diagnostic data may comprise a line plot of one or more identifiable parameters, as discussed in further detail below. In other examples, the diagnostic data may include two-dimensional or higher dimensional dot plots.

For example, in one instance, the cell attribute determination module 306 determines that the cells of the biological sample comprise small or large cell lymphoma based on the cell morphology presented. Then, the diagnostic data determination module 308 may independently determine a size distribution of the cells of the biological sample, confirming that the size distribution of the cells is consistent with small or large cell lymphoma. In another example, the cell attribute determination module 306 may determine that the cells of the biological sample are subject to left shift based on the cell morphology presented. The diagnostic data determination module 308 may likewise determine the presence of left shift based on a nucleus to cytoplasm ratio for neutrophils.

In some examples, diagnostic data is selected for presentation alongside extracted cell images. For example, the diagnostic data determination module 308 may access a look-up table to determine the type of diagnostic data to obtain based on the attributes determined by the cell attribute determination module 306. For example, a look-up table may indicate that for small or large cell lymphoma, the diagnostic data determination module 308 should determine size distribution. In another example, a look-up table may indicate that for left shift, the diagnostic data determination module 308 should determine a nucleus to cytoplasm ratio. In other examples, the diagnostic data determination module 308 may utilize methods or data structures other than a look-up table to determine the particular diagnostic data to obtain. Machine learning techniques may be used to train the diagnostic data determination module 308 to determine diagnostic data. In some examples, the diagnostic data determination module 308 may determine a disease state associated with the biological sample based on the determined diagnostic data. In these examples, the diagnostic data determination module 308 may also determine a confidence level associated with the determined disease state based on the determined diagnostic data.

Referring still to FIG. 3, the display module 310 may cause the output device 212 to display one or more images of the cells extracted by the cell image extraction module 304 and the diagnostic data determined by the diagnostic data determination module 308. In particular, the display module 310 may cause the output device 212 to display a mosaic image of cells that are relevant for interpretation based on the cell attributes determined by the cell attribute determination module 306, as discussed above.

The display module 310 may also cause the output device 212 to display the diagnostic data determined by the diagnostic data determination module 308, as discussed above, in an area adjacent to the displayed cells. This may allow a clinician to quickly view the cells of interest and the determined diagnostic data and confirm that a disease state indicated by the images of the cells matches the disease state indicated by the diagnostic data, thereby providing confidence to the clinician in the accuracy of the determinations made by the algorithms disclosed herein. In some examples, the display module 310 may cause the output device 212 to display the diagnostic data as a line plot associated with a parameter along with cutoff ranges, as discussed in further detail below.

In some examples, the display module 310 may also cause the output device 212 to display images of normal or references cells not having a condition or disease state. In some examples, the display module 310 may cause the output device 212 to display reference diagnostic data associated with cells not having a condition or disease state. A clinician may utilize the images of the reference cells and/or the reference diagnostic data to compare to the sample cell images and/or diagnostic data in order to assist with determining or confirming a diagnosis. Specific examples of what may be displayed by the display module 310 are disclosed in further detail below.

Referring still to FIG. 3, the attribute determination training module 312 and the diagnostic data determination training module 314 are discussed herein. In embodiments, the attribute determination training module 312 may train or refine the machine learning model utilized by the cell attribute determination module 306, as disclosed herein. In particular, as discussed above, both the cell attribute determination module 306 and the diagnostic data determination module 308 may determine a disease state associated with a biological sample and confidence levels associated with that determination.

In embodiments, if the diagnostic data determination module 308 determines a particular disease state associated with the biological sample with a first confidence level, and the cell attribute determination module 306 determines a particular disease state (either the same disease state or a different disease state) with a second confidence level that is lower than the first confidence level, this indicates that the diagnostic data determination module 308 is more confident in its determination of the disease state of the biological sample than the cell attribute determination module 306. As such, in this instance, the disease state determined by the diagnostic data determination module 308 and the associated cell images may be used as additional training data by the attribute determination training module 312 to refine the machine learning model used by the cell attribute determination module 306. Accordingly, the performance of the cell attribute determination module 306 may be improved for future analysis of biological samples.

Similarly, if the diagnostic data determination module 308 determines a particular disease state associated with the biological sample with a first confidence level, and the cell attribute determination module 306 determines a particular disease state with a second confidence level that is higher than the first confidence level, this indicates that the cell attribute determination module 306 is more confident in its determination of the disease state of the biological sample than the diagnostic data determination module 308. As such, in this instance, the disease state determined by the cell attribute determination module 306 and the associated cell images may be used as additional training data by the diagnostic data determination training module 314 to refine the machine learning model used by the diagnostic data determination module 308. Accordingly, the performance of the diagnostic data determination module 308 may be improved for future analysis of biological samples.

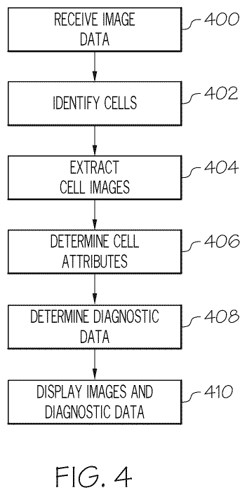

FIG. 4 depicts a flowchart of an example method for operating the digital microscope 100, according to the embodiments disclosed herein. At step 400, the image data reception module 300 receives image data captured by the digital microscope 100 as discussed above. In particular, the image data reception module 300 receives one or more images of a blood sample captured when illuminated by one or more wavelengths of light illuminated by the light source 102.

At step 402, the cell identification module 302 identifies the cells of the blood sample based on the image data received by the image data reception module 300, as discussed above. In particular, the cell identification module 302 may utilize machine learning techniques to define bounding boxes around individual cells in the one or more images received by the image data reception module 300.

At step 404, the cell image extraction module 304 extracts images of the cells identified by the cell identification module 302, as discussed above. In particular, the cell image extraction module 304 may extract individual images of each cell defined by a bounding box by the cell identification module 302.

At step 406, the cell attribute determination module 306 determines cell attributes of the cells identified by the cell identification module 302 based on the images of the cells extracted by the cell image extraction module 304, as discussed above. In particular, the cell attribute determination module 306 may use machine learning techniques to classify the cells based on cell type and/or attributes such as disease states or abnormalities.

At step 408, the diagnostic data determination module 308 determines diagnostic data associated with the blood sample of the images received by the image data reception module 300. In particular, the diagnostic data determination module 308 may determine particular diagnostic data based on the attributes determined by the cell attribute determination module 306.

At step 410, the display module 310 causes the output device 212 to display images of cells extracted by the cell image extraction module 304 and diagnostic data determined by the diagnostic data determination module 308.

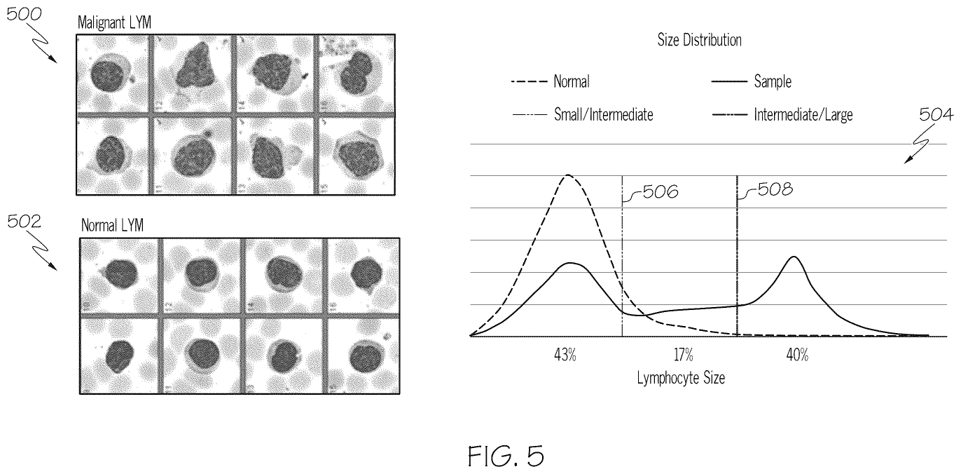

Examples of different disease states that may be indicated by the digital microscope 100 are now discussed. FIG. 5 shows an image that may be displayed by the output device 212 in one example. In the example of FIG. 5, a blood sample exhibiting large cell lymphoma is analyzed by the digital microscope 100. As shown in FIG. 5, the output device 212 outputs a first mosaic 500, a second mosaic 502, and a plot 504.

One method of identifying large cell lymphoma is separating lymphocytes into small, medium, and large cells. When the proportion of large lymphocytes in a lymphoma sample is above a specific threshold (e.g., 50%), the sample may be classified as large cell lymphoma.

In the example of FIG. 5, the first mosaic 500 comprises images of malignant lymphocytes from the analyzed blood sample identified by the cell identification module 302 and extracted by the cell image extraction module 304. The cell attribute determination module 306 may identify the cells as exhibiting large cell lymphoma. The second mosaic 502 comprises images of normal lymphocytes to be used as a reference.

The plot 504 shows a plot of lymphocyte size for the cells of the blood sample that may be determined by the diagnostic data determination module 308. In particular, the diagnostic data determination module 308 may determine that lymphocyte size should be plotted based on the large cell lymphoma determined by the cell attribute determination module 306. The plot 504 also shows lymphocyte size for a normal blood sample as a reference. Vertical lines 506 and 508 divide the plot 504 into small, intermediate, and large lymphocytes for easy reference. By presenting the mosaics 500 and 502 next to the plot 504, a clinician can quickly and easily see that the blood sample exhibits lymphoma both from the cell images and the size distribution of the cells of the blood sample.

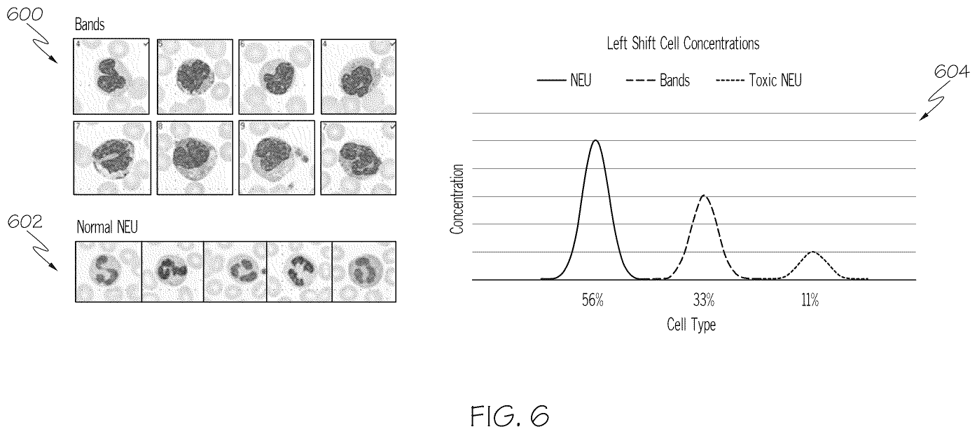

Turning now to FIG. 6, an example is shown for identifying acute inflammation in peripheral blood. In this situation, it is desirable to determine what portion of neutrophils present as immature (e.g., bands) and/or toxic as opposite to normal mature segmented neutrophils. In the example of FIG. 6, an image displayed by the output device 212 may comprise a first mosaic 600, a second mosaic 602, and a plot 604.

The first mosaic 600 may contain images of neutrophils presenting as bands identified by the cell identification module 302 and extracted by the cell image extraction module 304. The second mosaic 602 shows normal neutrophils as a reference. The plot 604 shows left shift concentrations for the blood sample determined by the diagnostic data determination module 308. In embodiments, a mosaic showing concentrations may show absolute counts or percentage counts.

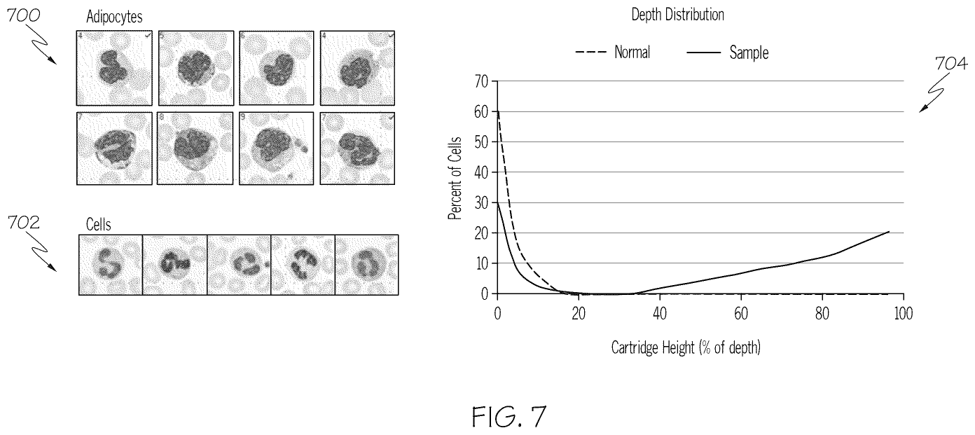

Turning now to FIG. 7, an example is shown for identifying adipocytes. The example of FIG. 7 shows a first mosaic 700 comprising adipocytes identified by the cell identification module 302 and extracted by the cell image extraction module 304. A second mosaic 702 contains images of normal cells as a reference. The example of FIG. 7 also shows a plot 704 of cell depth distribution determined by the diagnostic data determination module 308. Normal cell depth distribution is also plotted as a reference.

In other examples, the digital microscope 100 may identify mast cell tumors, hypogranularity, multinucleation, mitotic activity, anisocytosis, anisokaryosis, nuclear lemorphosm, nucleus to cytoplasm ratio, nucleus size or shape variability, nucleus shape (e.g., round, segmented), and other conditions. In other examples, the digital microscope 100 may allow for separating normal and degenerate neutrophils, and separating well granulated and poorly granulated mast cells.

It should now be understood that embodiments disclosed herein are directed to digital microscopy data visualization. By presenting images of cells of a blood sample and diagnostic data associated with the blood sample in the same image, a clinical pathologist may quickly identify disease states associated with the blood sample. By using a first algorithm to identify and image diseased cells and a second algorithm to determine the diagnostic data, the clinical pathologist can determine whether the disease state suggested by the cell images matches the disease state suggested by the diagnostic data. This may provide confidence to the clinical pathologist that the algorithms are operating properly and accurately diagnosing the condition associated with the blood sample.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method comprising:
receiving an image of a plurality of cells of a biological sample;
identifying, by a processor executing a first algorithm using image recognition machine- learning logic on the image, one or more cells of the plurality of cells as comprising one or more attributes associated with a first condition;
extracting individual images of the one or more identified cells;
determining, by the processor executing a second algorithm using second machine-learning logic, diagnostic data comprising one or more identifiable parameters associated with the plurality of cells;
determining whether the one or more identifiable parameters are associated with the first condition; and
displaying the individual images and the diagnostic data.

2. The method of claim 1, further comprising displaying one or more reference images of cells not having the first condition.

3. The method of claim 1, further comprising displaying reference diagnostic data associated with cells not having the first condition.

4. The method of claim 1, further comprising displaying a mosaic image of the one or more identified cells.

5. The method of claim 1, further comprising displaying one or more cutoff ranges of the diagnostic data.

6. The method of claim 1, wherein the first condition is large cell lymphoma, and the diagnostic data is a size distribution of lymphocytes in the biological sample.

7. The method of claim 1, wherein the first condition is acute inflammation in peripheral blood, and the diagnostic data is left shift concentration in the biological sample.

8. The method of claim 1, wherein the first condition is adipocytes, and the diagnostic data is depth distribution of cells in the biological sample.

9. The method of claim 1, further comprising:
determining a first confidence level associated with the one or more attributes;
determining a second confidence level associated with the one or more identifiable parameters;
determining whether the first confidence level is greater than the second confidence level;
in response to determining that the first confidence level is greater than the second confidence level, updating the second machine-learning logic; and
in response to determining that the second confidence level is greater than the first confidence level, updating the image recognition machine-learning logic.

10. The method of claim 9, wherein the biological sample is blood.

11. An apparatus comprising:
a processor and a non-transitory memory having stored therein instructions executable by the processor to cause the processor to:
receive an image of a plurality of cells of a biological sample;
identify, by executing a first algorithm using image recognition machine-learning logic on the image, one or more cells of the plurality of cells as comprising one or more attributes associated with a first condition;
extract individual images of the one or more identified cells;
determine, by executing a second algorithm using second machine-learning logic, diagnostic data comprising one or more identifiable parameters;
determine whether the one or more identifiable parameters are associated with the first condition; and
display the individual images and the diagnostic data.

12. The apparatus of claim 11, wherein the instructions, when executed, further cause the processor to display one or more reference images of cells not having the first condition.

13. The apparatus of claim 11, wherein the instructions, when executed, further cause the processor to display reference diagnostic data associated with cells not having the first condition.

14. The apparatus of claim 11, wherein the instructions, when executed, further cause the processor to display a mosaic image of the one or more identified cells.

15. The apparatus of claim 11, wherein the diagnostic data comprises a line plot of the one or more identifiable parameters.

16. The apparatus of claim 11, wherein the instructions, when executed, further cause the processor to display one or more cutoff ranges of the diagnostic data.

17. The apparatus of claim 11, wherein the first condition is large cell lymphoma, and the diagnostic data is a size distribution of lymphocytes in the biological sample.

18. The apparatus of claim 11, wherein the first condition is acute inflammation in peripheral blood, and the diagnostic data is left shift concentration in the biological sample.

19. The apparatus of claim 11, wherein the first condition is adipocytes, and the diagnostic data is depth distribution of cells in the biological sample.

20. The apparatus of claim 11, wherein the instructions, when executed, further cause the processor to:
determine a first confidence level associated with the one or more attributes;
determine a second confidence level associated with the one or more identifiable parameters;
determine whether the first confidence level is greater than the second confidence level;
in response to determining that the first confidence level is greater than the second confidence level, update the second machine-learning logic; and
in response to determining that the second confidence level is greater than the first confidence level, update the image recognition machine-learning logic.
